(19) United States
(12) Patent Application Publication
(10) Pub. No.: US 2004/0009235 A1
(43) Pub. Date: Jan. 15, 2004

(54) Nanometer grade hydroxyapatite composition for replenishing calcium

(76) Inventor: Jinghong Li, Changchun (CN)

Correspondence Address: KNOBBE MARTENS OLSON & BEAR LLP, 2040 MAIN STREET, FOURTEENTH FLOOR, IRVINE, CA, 92614, US

(21) Appl. No.: 10/460,281

(22) Filed: Jun. 12, 2003

(30) Foreign Application Priority Data
Jul. 9, 2002 (CN) 02123918.5

(51) Int. Cl. A61K 33/42; A61K 9/14; A61K 31/355; A01N 31/04; A61K 31/375

(52) U.S. Cl. 424/602; 514/168; 514/474; 514/458; 514/725

(57) ABSTRACT

The invention provides a hydroxyapatite composition for replenishing calcium, comprising 5 to 80 wt % hydroxyapatite with particle size of 5-800 nm and pharmaceutically acceptable excipient. The composition may comprise organic acid, such as citric acid, malic acid, starch, dextrin, sugar, Vitamin D, Vitamin A, Vitamin B, Vitamin C, and Vitamin E, and trace clement(s) selected from the group consisting of Sr, F, Fe, Zn, and Mn. The composition may be in solid preparation form such as powder, tablet, including coated tablet, capsule, and infusion (medicinal granules), and liquid preparation form such as oral liquid, injection solution, and beverage; and ion introduction type transdermal-preparation, etc. Furthermore the composition can be incorporated into any other foods. The composition of the invention can be made into different type of products including general type, children type, pregnant women type, liver and kidney type and climacteric type to meet the need of different people.

### RELATED APPLICATIONS

The present Application claims the benefit of priority of the Chinese application 02123918.5 filed Jul. 9, 2002, which is expressly incorporated herein by reference in its entirety.

### FIELD OF THE INVENTION

The present invention relates to a composition for replenishing calcium containing nanometer grade hydroxyapatite as active agent.

### BACKGROUND OF THE INVENTION

Calcium is one of the largest amount and the most important element contained in the body of human beings, which is important to maintain normal function of respiratory, nervous, digestive, endocrine, urinary, and immune system.

It is suggested from the current medical investigation that acalcerosis (deficiency of calcium) occurs commonly not only in developing countries but also in developed countries such as American, Europe, Japan, and the like. As to the Chinese, the deficiency of calcium is even a common social problem since the diet of the Chinese people is mainly composed of plant source. However, the calcium contained in plant source is difficult to be absorbed by human being. Acalcerosis is a relatively common problem in that too many people are considered to be acalcerotic. In China, up to 80 million people are diagnosed to be osteoporosis. Among 95 million children under 3 years old, the incidences of rachitis due to acalcerosis are 49% in the North and 24% in the South, respectively. In China, taking no account of those with acalcerosis in the youth and the prime of the life, the elderly, children, infants, and pregnant women that need to supplement calcium are up to 330 million. Furthermore, acalcerosis may induce many diseases. For children, acalcerosis is liable to result in rachitis, rheum, night cry, dysphoria, anorexia, constipation, pigeon breast, “0” or “X” type leg and the like. For pregnant women, acalcerosis may result in ache at the waist and back, numb hands and feet, muscle cramps, even severely result in antepartum syndrome of hypertension and postpartum odontoseisis, and affect the health of infant. Also, the old people are liable to suffer from acalcerosis. It has been known that the hypertension, arrhythmia, diabetes, and helcosis of many middle-aged persons and the elderly are relevant to aberration of calcium metabolism.

There are currently many calcium-replenished preparations in the market. For example, CN 95108344.9 disclosed a preparation containing calcium oxide, calcium hydroxide, and biological calcium carbonate, which has an advantage of simple preparation. However, the particle size is in μm order, which is too large to be absorbed by a human being. CN 98117505.8 disclosed a preparation using calcium acetate, calcium lactate, calcium citrate, calcium gluconate, calcium threonate, and complex decalcifying agent for hydrocarbon oil. However, the preparation can not achieve any satisfactrory effect due to the deficiency of content of calcium. CN 93106178.4 disclosed an calcium extraction from a halobios such as oyster, conch and the like. The producing process thereof is simple and the cost therefor is low. However, the product may contain pollutants from the ocean. Furthermore, the content of calcium is not sufficient and the particle size is too large. Therefore, it is difficult to satisfy the absorption of calcium of a human being. It is proved that the maximum bio-availability of calcium-replenished preparations available in the international market is only 30%. Furthermore the components of such preparations are complicated. For example, these preparations contain components effecting adversely absorption of calcium, such as oxalic acid, etc. Particularly, these preparations contain large amount of calcium salt, which will irritate significantly stomach due to the strong alkalinity thereof. Therefore, it is necessary to develop a series of calcium-replenished preparations with high calcium content, low side effects, ability to be absorbed by a human body, and satisfaction effect to meet the need of different people. Hydroxyapatite, i.e. Ca₁₀(PO₄)₆(OH)₂, is an inorganic component constituting hard tissue of vertebrates and human beings. For example, the skeleton of an adult is composed of 65% inorganic substances including hydroxyapatite and 35% organic substances. Particularly, the content of hydroxyapatite in the surface of enamel of tooth of human being is up to 95%.

### SUMMARY OF THE INVENTION

The present invention provides a nanometer-grade hydroxyapatite composition for replenishing calcium, wherein the particle size of hydroxyapatite is 5-800 nm. It is the best vehicle for biologically active calcium, which is easy to be absorbed by a human. Furthermore, it has an advantage of high bioavailability, no irritation of stomach and intestine, and no side effects. Particularly, according to the invention, the particle size of hydroxyapatite is in nanometer grade, which is easier to be absorbed by a human. The rate of absorption of the composition of the invention is much higher than those in the prior art, thus the efficacy thereof is improved significantly.

### DETAILED DESCRIPTION OF THE PREFERRED EMBODIMENT

The invention provides a hydroxyapatite composition for replenishing calcium, comprising hydroxyapatite with particle size of 5-800 nm and a pharmaceutically acceptable excipient.

According to the invention, the hydroxyapatite composition comprises 5 to 80 wt % hydroxyapatite with particle size of 5-800 nm, 0 to 30 wt % organic acid, 4 to 50 wt % starch, 0 to 40 wt % dextrin, 0 to 30 wt % sugar, 0 to 10 wt % Vitamin D, Vitamin A, Vitamin B, Vitamin C, and Vitamin E, and 0 to 5 wt % trace element.

According to one aspect of the invention, the organic acid is citric acid and/or malic acid.

According to another aspect of the invention, the trace element is one and more selected from the group consisting of Sr, F, Fe, Zn, and Mn.

According to still another aspect of the invention, the form of composition for replenishing calcium comprises solid preparations, liquid preparations, and ion introduction type transdermal preparations. In an embodiment of the invention, the solid preparation is selected from the group consisting of powder, tablet, coating tablet, capsule, and infusion. In another embodiment of the invention, the liquid preparation is selected from the group consisting of oral liquid, injection solution, and beverage. In addition, the composition according to the invention can be incorporated into any other foods.

The composition of the invention can be made into different type of products including general type, children type, pregnant women type, liver and kidney type and climacteric type to meet with the need of different people. In addition to hydroxyapatite as a major component, the composition of the invention comprises additionally Vitamin A, B, C, D, E, and trace element, such as Fe and Zn to improve nourishing and health care effect. The composition of the invention is a nanometer grade calcium preparation, which is easier to be absorbed by a human. The rate of absorption of the composition of the invention is much higher than those in the prior art, thus the efficacy thereof is improved significantly.

The synthesis methods of nanometer grade hydroxyapatite include physical methods and chemical methods. The physical methods include mechanical disintegration, for instance, ball milling, oscillating ball milling, oscillating milling, stirring milling, colloidal milling, nano-crush streaming and so on; evaporation agglomeration, which includes vacuum evaporation on rheologic oil surface, plasma heating evaporation, laser heating evaporation, electron beam heating evaporation, electric arc discharge heating evaporation, high frequency inducing heating evaporation, solar furnace heating evaporation and so on; ionic sputtering; freezing drying; spark discharging and explosion sintering. The chemical method includes gas-phase chemical reaction method, for instance, gas-phase decomposition, gas-phase synthesis, gas-solid reaction; deposition method, for instance, codeposition, hydrolysis deposition, hydrothermal; aerosol spray pyrolysis method; solvent evaporation decomposition method; sol-gel method; electrochemical synthesis method and radiochemical synthesis method.

The hydroxyapatite used in the invention has affinity for most of human proteins, thus has excellent biological compatibility. After implantation, it is safe and nontoxic, and furthermore promotes bone growth. Therefore it is well-known that hydroxyapatite is the star of biological ceramic material for medical use. At the same time, phosphorus itself is a nutritious element for all kinds of organisms and human, which has excellent biological compatibility, biological activity, and safety to soft and hard tissues. Therefore, from the point of view of biology, hydroxyapatite is the most suitable material to replace hard tissue of an organism and a new and efficient agent for replenishing calcium easy to be absorbed.

Since the method for preparing nanometer grade hydroxyapatite is wellknown to those skilled in the art, it is not described in detail in the following examples.

### EXAMPLE 1

To 120 L mixer 60 kg hydroxyapatite with particle size of 100±5 nm was added, then 3 kg citric acid, 4 kg malic acid, 3 kg starch, 3 kg sugar, 100 g strontium, 165 g fluorine, 150 g iron, 170 g zinc, and 50 g manganese were added respectively. The mixtures were blended sufficiently, then pressed into tablets with tablet weight of 0.5 g per tablet with a tablet press. Thus the nanometer-grade hydroxyapatite composition of the present invention was obtained. The absorption rate of the composition for human body was up to 85%.

### EXAMPLE 2

To 120 L mixer 65 kg hydroxyapatite with particle size of 30±5 nm was added, then 2 kg citric acid, 3 kg malic acid, 30 kg starch, 100 g strontium, 165 g fluorine, 150 g iron, and 170 g zinc were added respectively. The mixtures were blended sufficiently, then pressed into tablets with tablet weight of 0.5 g per tablet with a tablet press. Thus the nanometer-grade hydroxyapatite composition of the present invention was obtained. The absorption rate of the composition for human body was up to 90%.

### EXAMPLE 3

To 120 L mixer 70 kg hydroxyapatite with particle size of 30±5 nm was added, then 2 kg citric acid, 2 kg malic acid, 14 kg starch, 200 g Vitamin D, 200 g Vitamin B, 200 g Vitamin C, and 200 g Vitamin E were added respectively. The mixtures were blended sufficiently, then pressed into tablets with tablet weight of 0.5 g per tablet with a tablet press. Thus the nanometer-grade hydroxyapatite composition of the present invention was obtained. The absorption rate of the composition for human body was up to 90%.

### EXAMPLE 4

To a 200 L conventional mixer 70 kg hydroxyapatite with particle size of 300±5 nm was added, then 15 kg citric acid, 17 kg malic acid, 5 kg starch, 2 kg sugar, 100 g Vitamin D, 100 g Vitamin A, 100 g Vitamin B, and 100 g Vitamin C were added respectively. The mixtures were blended, then the powder of the nanometer-grade hydroxyapatite composition of the present invention was obtained. The absorption rate of the composition for human body was up to 82%.

### EXAMPLE 5

To a 200 L conventional mixer 3 kg hydroxyapatite with particle size of 100±5 m was added, then 8 kg citric acid, 8 kg malic acid, 20 kg starch, 19 kg sugar, 100 g Vitamin D, 100 g Vitamin A, 100 g Vitamin B, and 100 g Vitamin C 100 g Vitamin E, 100 g strontium, 165 g fluorine, 150 g iron, and 170 g zinc were added respectively. The mixtures were blended sufficiently, then the powder of the nanometer-grade hydroxyapatite composition of the present invention was obtained. The absorption rate of the composition for human body was up to 90%.

### EXAMPLE 6

To a 200 L conventional mixer, 100 kg hydroxyapatite with particle size of 100±5 nm was added, then 10 kg citric acid, 5 kg starch, 5 kg Vitamin D, 5 kg Vitamin A, 5 kg Vitamin C, 150 g iron, 170 g zinc, and 100 g manganese were added respectively. The mixtures were blended sufficiently, then the powder of the nanometer-grade hydroxyapatite composition of the present invention was obtained. The absorption rate of the composition for human body was up to 90%.

### EXAMPLE 7

To a 120 L mixer 70 kg hydroxyapatite with particle size of 200±5 nm was added, then 10 kg malic acid, 8 kg starch, 200 g Vitamin D, 200 g Vitamin A, 200 g Vitamin B, 200 g Vitamin C, and 200 g Vitamin E were added respectively. The mixtures were blended sufficiently, then pressed into tablets with tablet weight of 0.5 g per tablet with a tablet press. Thus the nanometer-grade hydroxyapatite composition of the present invention was obtained. The absorption rate of the composition for human body was up to 85%.

### EXAMPLE 8

To a 200 L conventional mixer 80 kg hydroxyapatite with particle size of 150±5 nm was added, then 15 kg starch, 6 kg sugar, 100 g Vitamin D, 100 g Vitamin A, 100 g Vitamin B, 100 g Vitamin C, 100 g Vitamin E, 1500 g strontium, 1000 g fluorine, 1500 g iron, 1500 g zinc, and 1500 g manganese were added respectively. The mixtures were blended, then the powder of the nanometer-grade hydroxyapatite composition of the present invention was obtained. The absorption rate of the composition for human body was up to 90%.

### EXAMPLE 9

As one of the preparation method for the general type of capsule for replenishing calcium, 6 kg hydroxyapatite with particle size of 200±5 nm was mixed with 600 g starch and 2.4 kg dextrin as adhesive to prepare soft wood, and therefore particles were prepared. The particles were dried at 100° C. for 8 hrs, and screened through a sieve of 60 mesh. Then 60 g citric acid was added. Under the condition of light and moisture proofing, i.e. at temperature lower than 28° C. and humidity lower than 40%, 0.03 g Vitamin D3 was added, and mixed repeatedly through a sieve using a method of increase by equivalent degrees. The mixtures were mixed with a high-speed mix pelletizer sufficiently, and encapsulated in suitable capsules, and sealed with aluminium foil or packaged into bottles with desiccant, and preserved under lucifuge condition. The absorption rate of the composition for human body was up to 85%.

### EXAMPLE 10

As one of the preparation method for the general type of capsule for replenishing calcium, 6 kg hydroxyapatite with particle size of 100±5 nm was mixed with 900 g starch and 1 kg dextrin as adhesive to prepare soft wood, and therefore particles were prepared. The particles were dried at 100° C. for 8 hrs, and screened through a sieve of 60 mesh. Then 60 g citric acid was added. Under the condition of light and moisture proofing, i.e. at temperature lower than 28° C. and humidity lower than 40%, 0.03 g Vitamin C was added, and mixed repeatedly through a sieve using a method of increase by equivalent degrees. The mixtures were further mixed with a high-speed mix pelletizer sufficiently, and encapsulated in suitable capsules, and sealed with aluminium foil or packaged into bottles with desiccant, and preserved under lucifuge condition. The absorption rate of the composition for human body was up to 90%.

### EXAMPLE 11

2 kg hydroxyapatite with particle size of 250±10 nm was added to a container with 50 L distilled water. Then 800 g citric acid was added, stirred to make them dissolved completely. Then, 200 g starch, 500 g zinc gluconate, and 8 g ferrous lactate were added, and stirred homogeneously, and then charged to 10 mL bottles, sealed, and preserved after sterilization. Thus the oral liquid of the nanometer grade hydroxyapatite composition of the present invention were obtained. The absorption rate of the composition for human body was up to 90%.

### EXAMPLE 12

2 kg hydroxyapatite with particle size of 100±5 nm was added to a container with 50 L distilled water. Then 300 g citric acid was added, stirred to make them dissolved completely. Then, 100 g starch, 500 g zinc gluconate, and 50 g ferrous lactate were added, and stirred homogeneously, and then charged to 10 mL bottles, sealed, and preserved after sterilization. Thus the oral liquid of the nanometer grade hydroxyapatite composition of the present invention were obtained. The absorption rate of the composition for human body was up to 90%.

What is claimed is:

1. A composition for replenishing calcium, comprising hydroxyapatite with particle size of between about 5 nm and about 800 nm and a pharmaceutically acceptable carrier.

2. The composition of claim 1, comprising between about 5 wt % and about 80 wt % hydroxyapatite with particle size of between about 5 and about 800 nm, and further comprising between about 0 wt % and about 30 wt % organic acid, between about 4 wt % and about 50 wt % starch, between about 0 wt % and about 40 wt % dextrin, between about 0 wt % and about 30 wt % sugar, between about 0 wt % and about 10 wt % vitamin, and between about 0 wt % and about 5 wt % trace element.

3. The composition of claim 2, wherein said organic acid is citric acid, malic acid, or a combination thereof.

4. The composition of claim 2, wherein said trace element is selected from the group consisting of: Sr, F, Fe, Zn, and Mn.

5. The composition of claim 2, wherein said vitamin is selected from the group consisting of: Vitamin D, Vitamin A, Vitamin B, Vitamin C, and Vitamin E.

6. The composition of claim 2, wherein said composition is in a form selected from: solid preparation, liquid preparation, and ion introduction type transdermal preparation.

7. The composition of claim 6, wherein said solid preparation is selected from the group consisting of: a powder, a tablet, a coating tablet, a capsule, and an infusion.

8. The composition of claim 6, wherein said liquid preparation is selected from the group consisting of: an oral liquid, an injection solution, and a beverage.

9. The composition of claim 1, wherein said composition is in a form selected from: solid preparation, liquid preparation, and ion introduction type transdermal preparation.

10. The composition of claim 9, wherein said solid preparation is selected from the group consisting of: a powder, a tablet, a coating tablet, a capsule, and an infusion.

11. The composition of claim 9, wherein said liquid preparation is selected from the group consisting of: an oral liquid, an injection solution, and a beverage.
